(19) United States
(12) Patent Application Publication
(10) Pub. No.: US 2008/0295831 A1
(43) Pub. Date: Dec. 4, 2008

(54) Inhaler vaporizer

(76) Inventors: Oswald C. Svehaug, Chula Vista, CA (US); Juana Svehaug, Chula Vista, CA (US)

Correspondence Address: RICHARD D. CLARKE;LAW OFFICE OF RICHARD D. CLARKE, 3755 AVOCADO BLVD., #1000, LA MESA, CA, 91941-7301, US

(21) Appl. No.: 11/810,060

(22) Filed: Jun. 4, 2007

(51) Int. Cl. A61M 16/00

(52) U.S. Cl. 128/203.15

(57) ABSTRACT

An inhaler vaporizer consists of two parts, the first part consisting of a funnel with a top portion, a “Y” portion and a disc portion made of a porous material or a non-porous material with a multiplicity of small holes. This part is inserted into a main body portion consisting of a main body, mouthpiece and base. The internal “Y” portion is sealed to the main body portion by an O-ring. Ethyl alcohol is poured into the main body of the inhaler vaporizer, and air is drawn into the inhaler vaporizer by applying suction through the mouthpiece. This air is drawn into the “Y” cone, passes through the “Y” stem tubular portion to the bottom of the body portion of the inhaler vaporizer. The air is drawn through the alcohol, vaporizing the alcohol in the process and is then inhaled through the mouthpiece into the mouth, throat, and lungs where it interacts with affected tissue.

### FIELD OF THE INVENTION

The present inhaler vaporizer generally relates to the field of devices for the treatment of colds, sore throats, influenza, and pulmonary infections. More specifically, this inhaler vaporizer device deals with a means by which these and other infections may be treated using an inhaler vaporizer to employ alcohol and water vapor in a safe and effective manner.

### DESCRIPTION OF THE RELATED ART

Traditional treatment regimes for lung, throat, sinus and bronchial infections involve getting a prescription from a health professional, generally after the infection has become worse when nothing available on the market is effective. Even with the intervention of health professionals at this time, the treatment of such infections can be lengthy and generally entails lengthy bed rest. Not only is this a health concern, but a monetary concern as well because prescription drugs may be expensive and the amount of time lost during the incubation of such infections can affect income, both to the patient and to family and personnel at work.

There is a vast market for a simple, cost effective method of treating the onset of infections in such diseases without prescriptions from health personnel who are likely to give the advice to get bed rest, take analgesics and drink chicken soup. This economic and health threat needs to be effectively treated with a simple and effective treatment available at very little cost and with no expensive prescriptions.

This novel invention utilizes over the counter consumable alcohol such as ethanol, and an inhaler vaporizer delivering vaporized ethanol directly to affected tissues utilizing a simple, yet effective method and apparatus.

There is a need for a new type of effective over the counter treatment for sinus, bronchial and throat infections that is harmless, convenient and does not involve the usual means of delivering medications such as nebulizers, humidifiers or atomizers that is functional, durable, and inexpensive.

An advantage of the inhaler vaporizer is to provide a small, unobtrusive method of delivering alcohol fumes directly to affected tissues.

Another advantage is to provide a means of providing treatment of infected tissues lightweight and easy to carry.

Another advantage is to provide an inhaler vaporizer that uses an over the counter alcohol meant for internal consumption.

Another advantage is to provide a fast, inexpensive and compact means of treating affected tissues without posing a discomfort for those around you such as on airlines. Liquid used within the inhaler vaporizer is available on most airlines.

Another advantage is to supply an inhaler vaporizer that is solid and compact which will last for years without concern that the liquid used with the inhaler will ever become outdated.

Another advantage is to supply a means of treatment which will be gentle and effective for children as well as adults.

Still another advantage of this invention is to provide a novel means of treating affected tissues that has a pleasing aroma, especially for those around you.

The inhaler vaporizer has as its primary structure an upright oriented tubular body having a base for the tube. The internal “Y” shaped internal part, has a bottom portion which is a tube, and a funnel portion which fits snugly into the outer tubular body with an O-ring substantially excluding air. Air is allowed into the body of the inhaler vaporizer via the internal “Y” stem tubular portion. Suction on the mouthpiece draws air into the alcohol through a porous disc or non-porous disc with small holes which penetrate the disc. This suction within the body portion of the inhaler vaporizer causes microscopic droplets of alcohol to be released into the mouth, and alcohol vapors into the lungs, and throat as the suction is applied to the inhaler vaporizer.

The internal “Y” shaped inner portion allows air to be sucked into the central stem of the removable inner portion of the inhaler or vaporizer. This air is drawn up the channels surrounding the stem of the internal “Y” portion through a small amount of alcohol. After becoming saturated with microscopically sized droplets of alcohol and alcohol fumes, the air is drawn into the mouth through the mouthpiece. The alcohol saturated air is delivered to the mouth, throat and lungs via suction caused by sucking on the mouthpiece. The disk shaped “Y” top end seals itself to the body of the external portion of the unit by means of an O-ring.

On both the first and second alternate embodiments of the inhaler vaporizer, the main structure of both the internal portion and external main body are the same with the exception of the disc through which the air passes prior to exiting through the mouth piece. The primary structural differentiation between them is the construction of the internal disc.

The internal disc is made up to non-porous material with very small holes piercing the body of the disc, allowing small bubbles of alcohol laden air to exit through the mouthpiece.

The alternate embodiment of the internal disc is made of a porous material through which air passes causing the formation of microscopic bubbles of alcohol laden air.

The above description, together with the objects of the invention and the various features of novelty that characterize the inhaler vaporizer are pointed out with particularity in the claims annexed to and forming a part of this disclosure. For a better understanding of the inhaler vaporizer, its operating advantages and the specific advantages attained by its uses, reference should be made to the accompanying drawings and descriptive matter in which there are illustrated preferred and alternate embodiments of the application.

### BRIEF DESCRIPTION OF THE DRAWINGS

The accompanying drawings, which are incorporated in and form a part of this specification, illustrate embodiments of the invention and together with the description, serve to explain the principals of this invention.

FIG. 1 is a front cross sectional view of the first embodiment of the novel inhaler vaporizer, constructed in accordance with the present invention;

FIG. 2 is a side elevational view of the first embodiment of the novel inhaler vaporizer, constructed in accordance with the present invention;

FIG. 3 is a side elevational view of the removable internal portion of the first embodiment of the novel inhaler vaporizer with porous disc, constructed in accordance with the present invention;

FIG. 4 is a side elevational view of the second embodiment of the internal removable portion as it would be if separated from the external portion of the second embodiment of the present invention with non-porous disc having a number of small holes extending through the disc, constructed in accordance with the present invention;

FIG. 5 is a side elevation of the external body portion of the novel inhaler vaporizer, constructed in accordance with the present invention;

FIG. 6 depicts a cross-sectional view of the first embodiment of the novel inhaler vaporizer, constructed in accordance with the present invention;

FIG. 7 depicts a top view of the first embodiment of the inhaler vaporizer, constructed in accordance with the present invention;

FIG. 8 depicts a cross sectional view of the second embodiment of the novel inhaler vaporizer, constructed in accordance with the present invention; and

FIG. 9 depicts a top view of the second embodiment of the inhaler vaporizer, constructed in accordance with the present invention.

### DETAILED DESCRIPTION OF THE PREFERRED EMBODIMENT

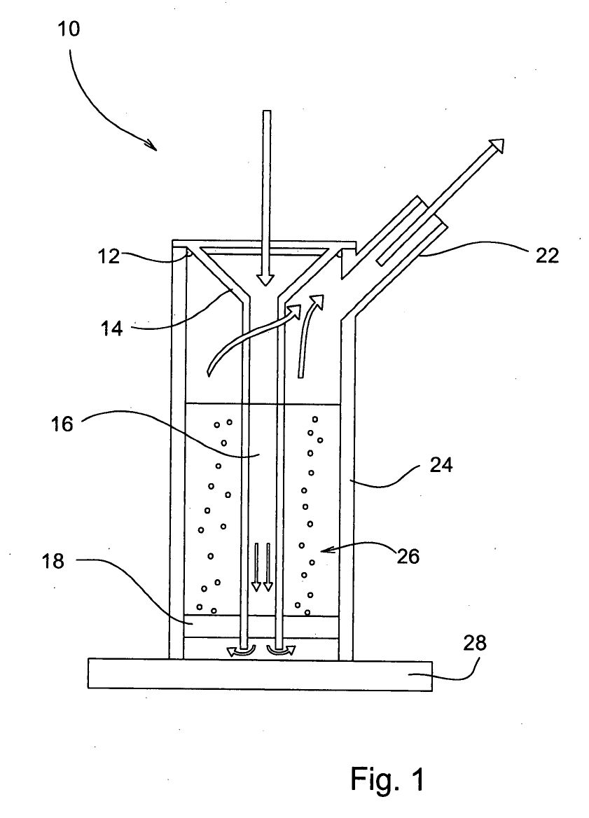

Referring now to the drawings, wherein similar parts are identified by like reference numerals, FIG. 1 shows a front cross sectional view of the first embodiment of the novel inhaler vaporizer 10 with an o ring 12 sealing the perimeter of the funnel cone 14 and the of the body 24 of the inhaler vaporizer 10. The air is drawn into the funnel stem 16 and drawn into the main body 24 of the inhaler vaporizer 10 when suction is applied through the mouthpiece 22 of the inhaler vaporizer 10. The air is pulled through the disc 18 as suction is applied on the mouthpiece 22. The aerated alcohol liquid 26 forms miniscule droplets and vapors of the alcohol which is inhaled in through the mouthpiece 22.

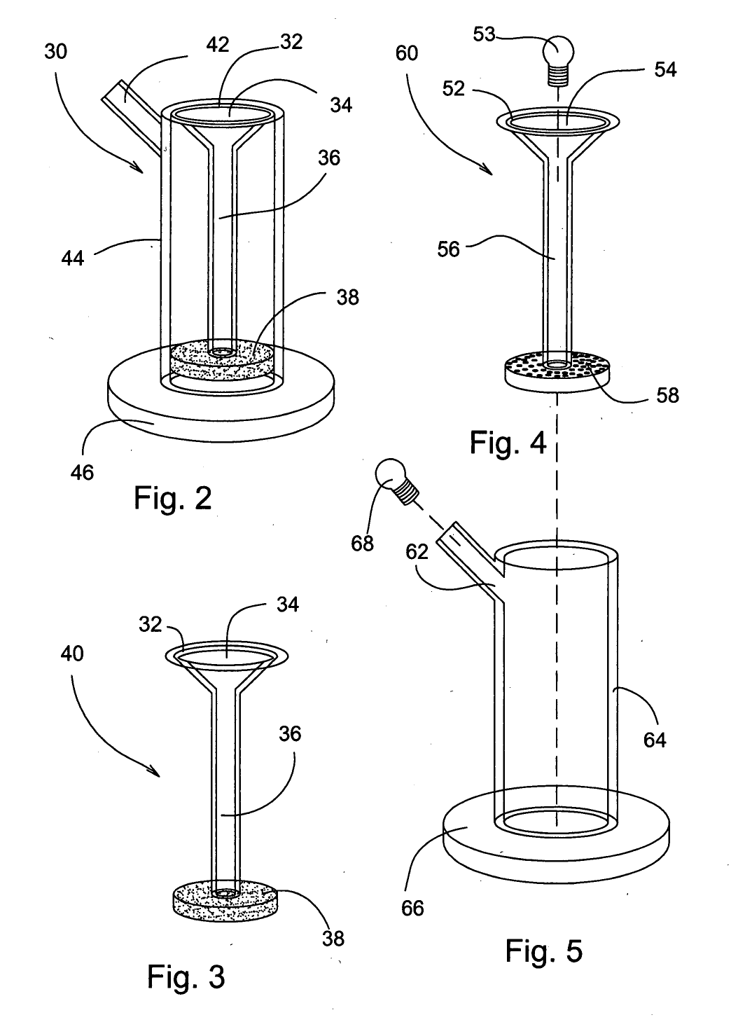

Referring now to FIG. 2, a side elevational view of the alternate embodiment 30, the mouth piece 42 is seen located on the side of the main body 44 of the inhaler vaporizer 30. The main body of the inhaler 44 is attached to the base of the body 46. The o ring 32 seals the funnel cone 34 to the main body 44 of the inhaler vaporizer 30. The funnel stem 36 passes through the porous disc 38.

Referring now to FIG. 3, a side elevational view of the y-shaped internal portion 40 of the first embodiment of the inhaler vaporizer. The gasket 32 is seen located on the funnel portion 34 of the y-shaped internal portion 40 of the inhaler vaporizer. The funnel stem 36 passes through the porous disc 38.

Referring now to FIG. 4, in the second embodiment of the inhaler vaporizer, the O-ring 52 is shown on the funnel cone 54 portion of the y-shaped internal portion 40 of the inhaler vaporizer. The funnel stem 56 passes through the disk of non-porous material 58 which has a plurality of small holes. An optional stopper plug 53 may be used to store the device, with or without liquid therein.

Referring now to FIG. 5, the mouthpiece 62 of the inhaler vaporizer attaches to the body 64 of the inhaler vaporizer which, in turn is attached to a base plate 66. An optional stopper plug 68 may be used to store the device, with or without liquid therein, and optionally with or without stopper plug 53.

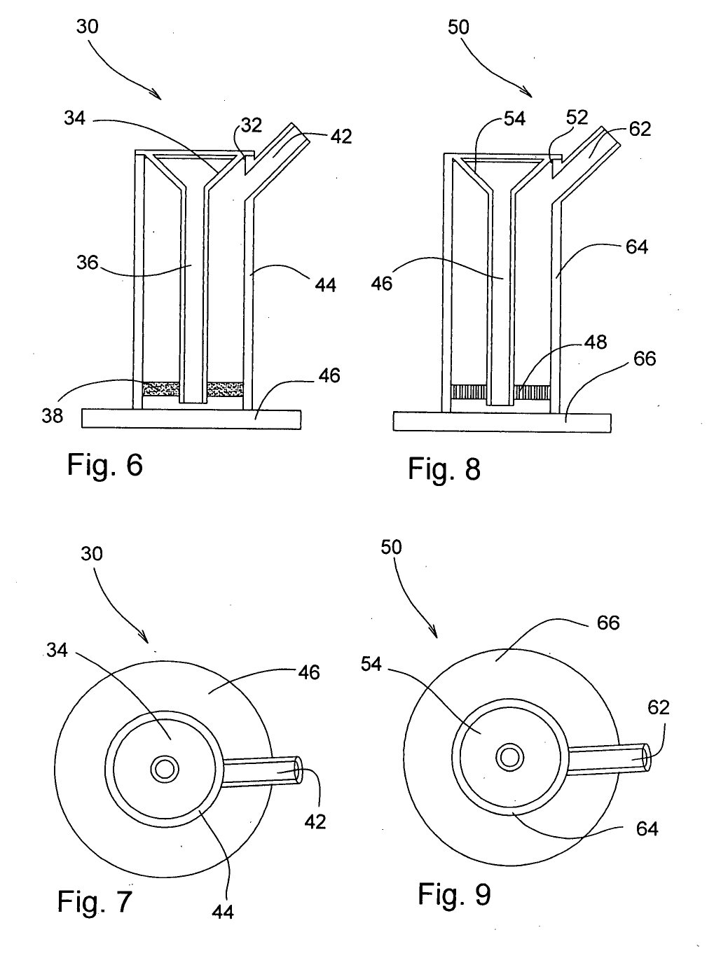

Referring now to FIG. 6, there is shown a side view of the first embodiment of the inhaler vaporizer 30. The O-ring 32 forms a seal between the funnel cone 34 and the main body 44 of the inhaler vaporizer 30. The funnel stem 36 passes through the disk composed of inert porous material 38.

As illustrated in FIG. 7, the main body 44 of the inhaler vaporizer 30 is attached to the base 46. The mouthpiece 42 is attached to the upper portion of the main body 44 of the inhaler vaporizer 30.

Referring now to FIG. 8, there is shown a second embodiment of the inhaler vaporizer 50 where the O-ring 52 serves to seal the funnel cone 54 to the main body 64 of the inhaler vaporizer 50. The stem of the funnel 46 passes through an opening in the non porous material with multiple holes 48. The main body 64 of the inhaler vaporizer 50 attaches to the base 66.

Referring now to FIG. 9, the base 66 supports the main body 64 of the inhaler vaporizer 50. The mouthpiece 62 is attached to the main body 64 of the inhaler vaporizer 50. The optimal disinfectant alcohol containing liquid would be between 50% to 50% alcohol (ethanol) to water and 65% to 35% alcohol (ethanol) to water. This range yields the best disinfecting action. Many commonly available alcoholic beverages are within this range, most notably whiskey.

The inhaler vaporizer embodiments 10, 30, and 50 as represented herein are designed to treat symptoms of common colds and nasal and throat infections but may be utilized as a preventative treatment of tissues after exposure to common infections as well. A communicable disease as such as tuberculosis, which can be contagious in small enclosed areas such as airplanes, may be prevented from infecting passengers by their use of the present invention. Treatment with the inhaler vaporizer device may act reduce or eliminate the tuberculosis bacillus.

The inhaler vaporizer 10, 30, and 50 shown in the drawings and described in detail herein disclose arrangements of elements of particular construction and configuration for illustrating preferred embodiments of structure and method of operation of the present design. It is to be understood however, that elements of different construction and configuration and other arrangements thereof, other than those illustrated and described may be employed for providing a inhaler vaporizer 10, and 50 in accordance with the spirit of this invention, and such changes, alternations and modifications as would occur to those skilled in the art are considered to be within the scope of this invention as broadly defined in the appended claims.

Furthermore, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

We claim:

1. An inhaler vaporizer device comprising:
a) a main vessel body, a mouthpiece portion and a base; and
b) a funnel with a Y-shaped top portion, a tubular portion extending downward, and a lower disc portion made of a non-porous material having a plurality of holes therein;
whereby said main vessel body is partially filled with an alcohol containing liquid and a user inhales air through said mouthpiece portion and the inhaled air passes through the alcohol containing liquid before entering the user's lungs.

2. The inhaler vaporizer device according to claim 1, wherein said non-porous material having a plurality of holes is replaced with a porous material having no holes.

3. The inhaler vaporizer device according to claim 1, wherein said main vessel body includes an O-ring to make an airtight seal between said main vessel body and said funnel Y-shaped portion.

4. The inhaler vaporizer device according to claim 1, wherein said alcohol containing liquid includes about 50% to 65% ethanol and about 50% to 35% water.

5. The inhaler vaporizer device according to claim 1, wherein said alcohol containing liquid includes a commonly available alcoholic beverage.

6. The inhaler vaporizer device according to claim 4, wherein said commonly available alcoholic beverage includes whiskey.

7. A method for making an inhaler vaporizer device, comprising the steps of:
a) providing a main vessel body, a mouthpiece portion and a base; and
b) providing a funnel with a Y-shaped top portion, a tubular portion extending downward, and a lower disc portion made of a non-porous material having a plurality of holes therein;
c) filling said main vessel body with an alcohol containing liquid; and
d) press fitting said funnel portion onto said main vessel body whereby said lower disc portion is submerged within said alcoholic liquid;
whereby said main vessel body being partially filled with an alcohol containing liquid, a user inhales air through said mouthpiece portion and the inhaled air passes through the alcohol containing liquid before entering the user's lungs.

8. The method for making an inhaler vaporizer device according to claim 7, wherein said step of providing a funnel with a Y-shaped top portion, a tubular portion extending downward, and a lower disc portion made of a non-porous material having a plurality of holes therein, is replaced with the step of providing a funnel with a Y-shaped top portion, a tubular portion extending downward, and a lower disc portion made of a porous material having no holes therein.

9. The method for making an inhaler vaporizer device according to claim 7, wherein said step of providing a main vessel body includes the step of providing a main vessel body wherein said main vessel body includes an O-ring to make an airtight seal between said main vessel body and said funnel Y-shaped portion.

10. The method for making an inhaler vaporizer device according to claim 7, wherein said step of filling said main vessel body with an alcohol containing liquid includes the step of filling said main vessel body with an alcohol containing liquid wherein said alcohol containing liquid includes about 50% to 65% ethanol and about 50% to 35% water.

11. The method for making an inhaler vaporizer device according to claim 7, wherein said step of filling said main vessel body with an alcoholic liquid includes the step of providing a commonly available alcoholic beverage.

12. The method for making an inhaler vaporizer device according to claim 11, wherein said step of providing a commonly available alcoholic beverage includes providing whiskey.

13. A method for using the inhaler vaporizer device as constructed according to claim 7, comprising the steps of:
a) filling said main vessel body with an alcohol containing liquid;
b) press fitting said funnel portion onto said main vessel body whereby said lower disc portion is submerged within said alcoholic liquid;
c) inhaling air through said mouthpiece portion whereby the inhaled air passes through the alcohol containing liquid before entering the user's lungs.

14. The method for using an inhaler vaporizer device according to claim 13, wherein said step of filling said main vessel body with an alcohol containing liquid includes the step of filling said main vessel body with an alcohol containing liquid having about 50% to 65% ethanol and about 50% to 35% water.

15. The method for using an inhaler vaporizer device according to claim 13, wherein said step of filling said main vessel body with an alcohol containing liquid includes the step of filling said main vessel body with a commonly available alcoholic beverage.

16. The method making an inhaler vaporizer device according to claim 15, wherein said step of filling said main vessel body with a commonly available alcoholic beverage includes the step of filling said main vessel body with whiskey.
